SEQUENCE LISTING (20 sequences)

SEQ ID NO: 1 - protein, 2392 aa, Myxococcus xanthus
VDPARLTRAWEGLLERYPLLAGAIRVEGTEPVIVPSGQVSAEVHEVPSVSDSALVATLRASAKVPFDLACGPLARLHLYSRSEHEHVLLLCFHHLVLDGASVAPLLDALRERYAGTEAKAGLLEVPIVAPYRAAVEWEQLAIGGDEGRRHLDYWRHVLATPVPPPLNLPTDRPRSATGLDSEGATHSQRVPTEQALRLREFARAQQVSLPTVLLGLYYALLHRHTRQDDVVVGIPTMGRPRAELATAIGYFVNVMAVRARGLGQHSFGSLLRHLHDSVIDGLEHAHYPFPRVVKDLRLSNGPEEAPGFQTMFTFQSLQLTSAPPRPEPRSGGLPELEPLDCVHQEGAYPLELEVVEGAKGLTLHFKYDARLYEADTVERMARQLLRAADQVADGVESPLSALSWLDDEERRTLLRDWNATATPFLEDLGVHELFQRQARETPDAMAVSYEGHSLSYQALDTRSREIAAHLKSFGVKPGALVGIYLDRSAELVAAMLGVLSAGAAYVPLDPVHPEDRLRYMLEDSGVVVVLARQASRDKVAAIAGASCKVCVLEDVKAGATSAPAGTSPNGLAYVIYTSGSTGRPKGVMIPHRGVVNFLLCMRRTLGLKRTDSLLAVTTYCFDIAALELLLPLCAGAQVIIASAETVRDAQALKRALRTHRPTLMQATPATWTLLFQSGWENAERVRILCGGEALPESLKAHFVRTASDVWNMFGPTETTIWSTMAKVSASRPVTIGKPIDNTQVYVLDDRMQPVPIGVPGELWIAGAGVACGYLNRPALTAERFVSNPFTPGTTLYRTGDLARWRADGEVEYLGRLDHQVKVRGFRIEMGEIEAQLAGHPSVKNCAVVAKELNGTSQLVAYCQPAGTSFDEEAIRAHLRKFLPDYMVPAHVFAVDAIPLSGNGKVDRGQLMARPVVTRRKTSAVHARSPVEATLVELWKNVLQVNEVGVEDRFFEVGGDSVLAAVLVEEMNRRFDTRLAVTDLFKYVNIRDMARHMEGATAQARTGATEPAREDTASERDYEGSLAVIGISCQLPGAADPWRFWKNLREGRDSVVAYRHEELRELGVPEEVLRDSRYVAVRSSIEDKECFDPHFFGLTARDASFMDPQFRLLLMHAWKAVEDAATTPERLGPCGVFMTASNSFYHQGSPQFPADGQPVLRTAEEYVLWVLAQAGSIPTMVSYKLGLKGPSLFVHTNCSSSLSALYVAQQAIAAGDCQTALVGAATVFPSANLGYLHQRGLNFSSAGRVKAFDAAADGMIAGEGVAVLVVKDAAAAVRDGDPIYCLVRKVGINNDGQDKVGLYAPSATGQAEVIRRLFDRTGIDPASIGYVEAHGTGTLLGDPVEVSALSEAFRTFTDRRGYCRLGSVKSNLGHLDTVAGLAGLIKTALSLRQGEVPPTLHVTQVNPKLELTDSPFVIADRLAPWPSLPGPRRAAVSAFGLGGTNTHAILEHYPRDSRPRERSQRSNAVRAVAPFSARTLEALKDNLRALLDFLEDPASAEVALADITYTLQVGRVAMPERMVVTASTRDELVEGLRRGIATVGGAHVGTVVDTSPSVDADARAVAEAWATGDSIDWDSLHGDVKPARVSLPTYQFAKERYGLSPAHSVANSSKTHPDAGVPLFVPTWQPWSEGASNASLALRHLVVLCEPLDALGAEGASALASTLADRRIEVVRTSSPSARLDARFMAHASAVFERVKALLSERLTAPVTLQVLVPEERDALALSGLGSLLRSVSQENPLVRGQLIRVQGSVSASALVDVLVKSARAGDVTDSRYHAGQLSRCEWREARVAKGDASRFWREDGVYVISGGTGALARLFVAEIGKRATRATVILVARASSAEAVDGGNGLRVRHLPVDVTQPNDVNAFVATVLREHGRIDGVIHAAGIRRDNYLLNKPVAEMQAVLAPKVVGLVNLDHATRELPLDFFVTFSSLAAFGNAGQSDYAAANGFMDGFAESRAALVNAGQRQGRTVSIRWPLWENGGMQLDSRSREVLMQRTGMAALGDEAGLGAFYRALELGSPGVAVWTGEAQRFRELSVSVSPAPPPHQVALDAVVSITEKVETKLKALFSEVTRYEERRIDARQPMERYGIDSIIITQMNQALEGPYNALSKTLFFEYRTLAEVSGYLAEHRAEESAKWVAAPGENSSSVIQEARPPRADATHRAPRADEPIAVIGMSGRYPGAENLTEFWERLSRGDDCITEIPPERWSLDGFFYPDKKHAAARGMSYSKWGGFLGGFADFDPLFFNISPREATSMDPQERLFLQSCWEVLEDAGYTRDSLAQRFGSAVGVFAGITKTGYELYGAELEGRDASVRPYTSFASVANRVSYLLDLKGPSMPVDTMCSASLTAVHMACEALQRGACVMAIAGGVNLYVHPSSYVSLSGQQMLS

SEQ ID NO: 2 - DNA, 7178 bp, Myxococcus xanthus
gtcgacccggcgaggctgacccgggcctgggaaggactgctcgaacggtatccgctgctcgctggcgcgattcgcgtcgaaggcacggagccggtcatcgtccccagtgggcaggtctccgccgaggtccacgaggttccatcggtctccgattcagcactggtggcgaccctgcgcgcctccgcgaaggtgccattcgatctcgcctgtggaccgctcgctcggctgcacctgtactcgcggtcggagcacgagcatgtcctgctgctgtgcttccaccacctggtgctcgatggggcatccgtggcgcccttgctcgacgccctccgggagcgttacgccgggaccgaggcgaaggcggggctgctcgaggttccgatcgtcgctccttaccgcgccgccgtggagtgggagcagctcgccattggaggcgatgagggacggcgccacctcgactactggcggcacgtgttggccacgcccgttcctccgccgttgaatcttccaacggaccggcctcgctccgccacggggctggactcggagggagcaacgcactcgcagagggtgcccaccgagcaagcattgcgactgcgcgagttcgctcgggcacagcaagtgagcctgccgaccgtcctgctcgggctctactacgccttgcttcatcggcacacgcgccaggacgacgtggtggtcggcatccccaccatggggcggccccgggcggaactggcgacggcgattgggtacttcgtcaacgtgatggccgtgcgcgcgcggggcctggggcagcactcgttcggctcgctgctgcgccacctccacgactcggtcatcgatggcctggagcatgcccactatcccttcccgcgagtggtgaaggacctccggctgtcgaatgggcccgaggaggcgcctggcttccagacgatgttcaccttccagagcctgcaactgacgagcgctccgccaaggccggagcccaggtcgggcgggttgccggagcttgagccgctcgactgcgtccatcaggaaggcgcctacccgctggagcttgaagtggtggagggcgccaagggcctcacgctgcatttcaagtacgacgcgcggctgtacgaggcggacacggtcgaacggatggcgcgtcagttgttgcgcgccgcggaccaggtcgcggatggggtggagtctccgctgagcgcactgtcgtggctcgacgacgaagagcgccgcacgcttctccgcgactggaatgccacggccacgccgttcctcgaggacctgggcgttcacgagctcttccagcggcaggcccgggagaccccagacgccatggctgtgagctacgaggggcactcgctcagctatcaggcgctggatacgcggagccgcgagattgcggcgcacctgaagagcttcggcgtcaagcctggggcgctcgtgggcatctacctggaccggtccgcggagctggtggcggcgatgctgggtgtgctgtccgctggcgcggcctacgtacccctggacccggtgcaccccgaggaccggctgcggtacatgctggaggacagtggcgtggtggtcgtgctggcccgtcaggcctcgcgggacaaggtcgccgccattgccggagcctcctgcaaggtgtgcgtgctggaggacgtcaaggctggagccacgtccgcgccggcgggaacctcaccgaacggacttgcctacgtcatctacacgtccgggagcacgggccggcccaagggcgtgatgattccccatcgcggggtggtcaacttcctcctgtgcatgcgcaggacgctgggcctgaagcgcacggattcgctgttggcggtcacgacgtactgcttcgacatcgcggcgctcgagctcctgcttccgctgtgtgcgggggcgcaggtcatcatcgcgtcggcggagacggttcgggatgcgcaggcgttgaagcgggcgctgcgcacccatcggcccacgttgatgcaggcgacgcccgcgacctggacactgttgttccagtctggctgggagaacgccgagcgggttcgaatcctctgcggtggagaagcgctgccggagtcgctcaaggcccacttcgttcgcaccgcgagcgacgtgtggaacatgttcgggcccaccgagacgaccatctggtcgacgatggcgaaggtctcggcctcgcgtccggtcaccattggaaagccgatcgacaacacgcaggtctacgtgctggacgaccggatgcagccggtgcccatcggtgtgccgggcgagctgtggattgcgggcgcgggcgtggcctgcggttacctcaaccggccggcgctgaccgccgagcgcttcgtttccaatccgttcacgccgggcacgacgctctaccggacgggggacctggcgcgctggcgcgctgacggtgaggttgagtacctggggcggctcgaccaccaggtgaaggtgcgcggcttccgcatcgagatgggggagattgaagcgcagttggccgggcatcccagcgtgaagaactgtgccgtggtggccaaggagctgaacggcacctcgcagctcgtcgcctactgtcagcccgcgggaacgagcttcgatgaggaagccatccgtgcacacctgcggaagttcctccccgactacatggtccccgcgcacgtcttcgcggtggatgcgattccgctgtcgggcaatggcaaggtggaccggggccagctgatggccaggccggtggtcacccggcggaagacatccgcggtccatgcccgttcgcctgttgaggccaccctcgtcgagctgtggaagaacgtgctccaggtcaacgaggtgggtgtcgaggatcgcttcttcgaagtggggggggactccgtgctggccgccgtgctggtggaggagatgaaccggcgcttcgacacgcggctcgccgtcaccgacctgttcaagtacgtcaatattcgcgacatggcgcgccacatggagggcgcgacggcgcaagcccgtactggggccaccgagccggctcgcgaggacaccgcgtcggagcgtgactacgagggcagcctggccgtcatcggcatctcctgtcagttgcccggagccgcggacccctggcgcttctggaagaacctgcgagagggcagggacagcgtggtggcgtaccgccatgaggaactgcgcgagctgggcgtgcccgaggaggtcctccgcgattcccgttacgtcgcggtccggtcgtccatcgaagacaaggagtgcttcgacccgcatttcttcggtctgacggcgcgggacgcgtccttcatggacccgcagttccgactgttgctgatgcacgcctggaaggcagtggaagacgcggcgacgacgcctgagcgcctgggaccgtgcggcgtcttcatgacggccagcaacagcttctatcaccagggctcgccgcaatttcctgcggacgggcagccggtcctccgcaccgccgaagaatacgtgctgtgggtgctggcccaggcaggctccatcccgacgatggtttcstacaagctcggcttgaaggggccgagcctgttcgtccacaccaactgctcgtcatccctgtccgcgctgtacgtggctcagcaggccatcgcagcgggagactgccagacggcgctggtgggggccgccacggtcttcccttcggcgaacttgggttatctgcaccagcgggggctcaacttctccagcgcggggcgggtcaaggccttcgacgccgcggcggacggcatgattgccggtgaaggtgtcgccgtgctggtggtgaaggacgccgcagcggcggtgcgcgatggcgacccaatctactgcctcgtgcggaaggtggggatcaacaacgacggccaggacaaggtgggtttatacgccccgagcgccaccgggcaggcggaggtcatccggcgtctgttcgaccggaccggcatcgaccctgcatcgattggctacgtcgaggcccatggcaccggaaccttgctgggtgaccctgtcgaggtctccgcgctgagcgaagccttccggaccttcaccgaccggcgcgggtactgccggctgggctcggtgaagtcgaacctgggccatctggacacagtggctggactggctgggctcatcaagacggcgctgagcctgcggcagggcgaagttcctccgacgctccatgtgacccaggtgaatccgaagctcgagctgacggattcgccgttcgtcatcgccgaccgtttggcgccgtggccgtccctgccgggaccgaggcgggcggccgtgagtgcgttcggccttggcgggacgaatacccacgccattctcgaacactacccgcgcgactcccgcccacgggagaggagccagcggtcgaacgcagtccgtgcggtggctccattctcggcgcgcaccctggaggcgttgaaggacaacctccgcgcgctgctcgacttcctggaggacccggcgtccgcggaggtggcgctcgcggacatcacctacacgttgcaggtcggccgggtcgcgatgcctgagcggatggtggtgactgcgtcgacgcgcgacgaattggtggagggactgcggcgaggcatcgcgacggtgggcggtgcccacgtgggaacggtggtcgatacgtcacccagcgtggatgccgatgctcgggcagttgcggaggcgtgggcgacgggcgactcgattgactgggattcgctgcacggtgacgtgaagcccgcccgtgtcagcctgcccacgtatcagttcgcgaaggagcgctacgggttgtcgcccgcgcactccgtggcgaattcctccaagacgcatcctgacgcgggtgtcccgctcttcgttccgacctggcagccgtggtctgagggcgcgtcaaatgcctcgttggcgctccggcacctggtggtgttgtgcgagcctcttgatgcgctgggggctgaaggtgcctccgcgctggcgagcacgctcgcggacaggcgcatcgaagtggtcaggacgtccagcccaagtgcgcggctggacgcgcggttcatggcgcatgcctcggcggtcttcgaacgcgtcaaggcgctgctgtcggagcgtctgaccgctcctgtgacattgcaggtgctggtgccagaggagcgggatgcgctggcactgagtggcctggggagcctgctgcgttcggtgtcgcaggagaatccgttggtccgggggcagctcatccgcgtccagggaagcgtctccgcatcggcgctggtggacgttctggtgaagtccgcgcgcgccggtgacgtcaccgattcgcggtaccacgcgggccagctttctcgctgtgagtggcgcgaggcacgtgtcgccaagggggacgcatcccgcttctggcgcgaagacggcgtctatgtgatttcaggaggaaccggcgccctggcccggctgttcgtcgccgaaatcgggaagcgcgcgacgcgggccaccgtcattctggttgcccgcgcatcctcggcggaggcggtggacggtgggaacgggctgcgcgtgcggcaccttcccgtggatgtcacccaaccgaacgacgtgaacgcctttgtcgctacggtgctgcgcgaacacgggcgcatcgacggtgtcatccatgcggcgggcatccgccgtgacaactacctgctcaacaagccggtggcggaaatgcaggcggtgctcgcgcccaaggtggtggggctcgtcaacctggaccacgccacccgcgagctgcccctggatttcttcgtcacgttctcgtccctggccgcgtttggaaacgccggtcagtcggactacgcggcggccaatggcttcatggacggattcgcggagtcccgagcggcgctcgtgaacgccggacagcggcagggccggacggtgtccatccgttggccgctctgggagaacggcgggatgcagctcgactcacggagccgtgaggtcttgatgcagcggaccgggatggccgcgctgggagacgaagcgggactgggggcgttctaccgggcgctggaactgggctcccctggtgtcgcggtgtggacgggggaggcccagaggtttcgtgaactctccgtgagtgtttcgcccgcaccgcctccgcatcaggtggcgttggacgccgtggtgtccatcaccgagaaggtcgagacgaagctgaaggcgctcttcagcgaggtcacgcgatacgaagagcgccgcatcgatgcccgccagccgatggagcgctatggcatcgactccatcatcatcacgcagatgaaccaagccctcgaagggccgtacaacgccctctcgaagacgctgttcttcgaataccggacgctcgcggaagtcagcgggtatctggccgagcaccgcgcggaagagagcgcgaagtgggtggcggcacctggagagaattcgtcttccgtcatccaggaggccaggccgccacgtgcggatgcgacgcaccgggcgcctcgcgccgacgagcccatcgccgtcattggcatgagcggccgttatcccggggcggagaacctgacggagttctgggagcgcctgagccgcggtgacgactgcatcaccgagattccgccagagcgctggtcgttggacgggttcttctacccggacaagaagcacgccgccgcgcgggggatgagctacagcaagtggggcggcttcctcggcggcttcgctgacttcgacccgctgttcttcaacatctcgccgcgtgaggcgacgagcatggacccgcaggagcgcttgttcctgcagagctgctgggaggtcctggaggacgcggggtacacccgggacagcctggcccagcgctttggcagcgcggtgggcgttttcgcgggaatcacgaagacgggctacgaactctacggcgcggagctggaaggacgagatgcctcggtccggccctatacgtcgtttgcgtctgttgccaaccgcgtctcgtatctgctcgacctgaaggggccgagcatgcccgtggacaccatgtgctcggcctcgctgacagccgtccacatggcttgcgaggcgctgcaacgaggcgcctgcgtcatggccatcgcgggtggagtgaatctctacgtccacccgtcgagctacgtcagcctgtccgggcagcagatgctgtcgac

SEQ ID NO: 3 - protein, 785 aa, Myxococcus xanthus
MKVVNKLLEKLPDVVAGKVPDVKLQDQDIKVPLAQGTFTEEKILPPKLAMHGFTLSFEATGEASIRNFNSLGDVDENGIIGEPSPESAEPGPRPQLLLGSDIGWMRYQVSARVKAAVSASLSFLASENQTELSVTLSDYRAHPLGQNMREAVRSDLSELRLMQATDLAKLTTGDAVAWHVRGALHTRLELNWADIFPTNLNRLGFLRGNELLALKTSAKAGLSARVSLTDDYQLSFSRPRAGRIQVAVRKVKSHEQALSAGLGITVELLDPATVKAQLGQLLEALLGPVLRDLVKKGTTAVEIMDGLVDKASKAKLDDNQKKVLGLVLERLGIDPQLADPANLPQAWADFKARVAESLENAVRTQVAEGFEYEYLRLSETSTLLEVVVEDVTAMRFHESLLKGNLVELLKWMKSLPAQQSEFELRNYLHATTLTRQQAIGFSLGLGSFELLKAKNVSKQSWVTQENFQGARRMAFLGRRGYEDKLLGTRGQWVVDLKADMTRFSPTPVASDFGYGLHLMLWGRQKKLSRKDLQQAVDDAVVWGVLDAKDAATVISTMQEDMGKHPIETRLELKMADDSFRALVPRIQTLELSRFSRALARALPWSEQLPRASAEFRRAVYAPIWEAYLREVQEQGSLMLNDLSPSRAAQIAKWYFQKDPTVRDLGKDLQLIESEWRPGGGNFSFAEVISKNPNTLMRCRNFVSGMVRLRRAIDERKAPDELRTVFGELEGMWTTGFHLRAAGSLLSDLAQSTPLGLAGVERTLTVRVADSEEQLVFSTARSTGAA

SEQ ID NO: 4 - protein, 529 aa, Myxococcus xanthus
MPSGCYGAASAFVLPPLPAMPQAPSDVSQVLLPFGGLVGREVDLDAFLQTLMDRIAITLQADRGTLWLLDPARRELFSRAAHLPEVSQIRVKLGQGVAGTVAKAGHAINVPDPRGEQRFFADIDRMTGYRTTSLLAVPLRDGDGALYGVLQVLNRRGEDRFTDEDTQRLTAIASQVSTALQSTSLYQELQRAKEQPQVPVGYFFNRIIGESPQLQAIYRLVRKAAPTDATVLLRGESGSGKELFARAVHVNGPRRDQPFIKVDCAALPATLIENELFGHERGAFTGADHRVPGKFEAASGGTVFIDEIGELPLPVQGKLLRVIQDREFERVGGTQAVKVDVRIVAATHRDLARMVAEGRFREDLYYRIKVVEVVLPPLRERGAEDIERLARHFVAAVARRHRLTPPRLSAAAVERLKRYRWPGNVRELENCIESAVVLCEGEILEEHLPLPDVDRAALPPPAAAQGVNAPTAPAPLDAGLLPLAEVERRHILRVLDAVKGNRTAAARVLAIGRNTLARKLKEYGLGDEP

SEQ ID NO: 5 - protein, 292 aa, Myxococcus xanthus
MRASQAEAPHSRRLTMEVRFHGVRGSIAVSGSRIGGNTACVEVTSQGHRLILDAGTGIRALGEIMMREGAPQEATLFFSHLHWDHVQGFPFFTPAWLPTSELTLYGPGANGAQALQSELAAQMQPLHFPVPLSTMRSRMDFRSALHARPVEVGPFRVTPIDVPHPQGCLAYRLEADGHSFVYATDVEVRVQELAPEVGRLFEGADVLCLDAQYTPDEYEGRKGVAKKGWGHSTMMDAAGVAGLVGARRLCLFHHDPAHGDDMLEDMAEQARALFPVCEPAREGQRLVLGRAA

SEQ ID NO: 6 - protein, 168 aa, Myxococcus xanthus
MPGPRCAENDWVALLVRVNHEKVAAAQLGKHGYEFFLPTYTPPKSSGVKAKLPLFPGYLFCRYQPLNPYRIVRAPGVIRLLGGDAGPEAVPAQELEAIRRVADSGVSSNPCDYLRVGQRVRIIEGPLTGLEGSLVTSKSQLRFIVSVGLLQRSVSVEVSAEQLEPITD

SEQ ID NO: 7 - protein, 79 aa, Myxococcus xanthus
MDKRIIFDIVTSSVREVVPELESHPFEPEDDLVGLGANSLDRAEIVNLTLEKLALNIPRVELIDAKTIGGLVDVLHARL

SEQ ID NO: 8 - protein, 420 aa, Myxococcus xanthus
MGPVGIEAMNAYCGIARLDVLQLATHRGLDTSRFANLLMEEKTVPLPYEDPVTYGVNAARPILDQLTAAERDSIELLVACTESSFDFGKAMSTYLHQHLGLSRNCRLIELKSACYSGVAGLQMAVNFILSGVSPGAKALVVASDLSRFSIAEGGDASTEDWSFAEPSSGAGAVAMLVSDTPRVFRVDVGANGYYGYEVMDTCRPVADSEAGDADLSLLSYLDCCENAFREYTRRVPAANYAESFGYLAFHTPFGGMVKGAHRTMMRKFSGKNRGDIEADFQRRVAPGLTYCQRVGNIMGATMALSLLGTIDHGDFATAKRIGCFSYGSGCSSEFFSGVVTEEGQQRQRALGLGEALGRRQQLSMPDYDALLKGNGLVRFGTRNAELDFGVVGSIRPGGWGRPLLFLSAIRDFHRDYQWIS

SEQ ID NO: 9 - protein, 325 aa, Myxococcus xanthus
MSSVATAVPLTARDSAVSRRLRITPSMCGQTSLFAGQIGDWAWDTVSRLCGTDVLTATNASGAPTYLAFYYFRIRGTPALHPGALRFGDTLDVTSKAYNFGSESVLTVHRICKTAEGGAPEADAFGHEELYEQPQPGRIYAETFNRWITRSDGKSNESLIKSSPVGFQYAHLPLLPDEYSPRRAYGDARARGTFHDVDSAEYRLTVDRFPLRYAVDVIRDVNGVGLIYFASYFSMVDWAIWQLARHQGRSEQAFLSRVVLDQQLCFLGNAALDTTFDIDVQHWERVGGGEELFNVKMREGAQGRDIAVATVKVRFDAASEGGRRG

SEQ ID NO: 10 - protein, 83 aa, Myxococcus xanthus
MTDEQIRGVVHQSIVRVLPRVRSNEIAGHLNLRELGADSVDRVEILTSILDSLRLQKTPLAKFADIRNIDALVAFLAGEVAGG

SEQ ID NO: 11 - protein, 374 aa, Myxococcus xanthus
MMQERGVALPFEDPVTNAVNAARPILDAMSPEARERIELLVTSSESGVDFSKSISSYAHEHLGLSRHCRFLEVKQACYAATGALQLALGYIASGVSPGAKALVIATDVTLVDESGLYSEPAMGTGGVAVLLGDEPRVMKMDLGAFGNYSYDVFDTARPSPEIDIGDVDRSLFTYLDCLKHSFAAYGRRVDGVDFVSTFDYLAMHTPFAGLVKAGHRKMMRELTPCDVDEIEADFGRRVKPSLQYPSLVGNLCSGSVYLSLCSIIDTIKPERSARVGMFSYGSGCSSEFFSGVIGPESVSALAGLDIGGHLRGRRQLTFDQYVELLKENLRCLVPTKNRDVDVERYLPLVTRTASRPRMLALRRVVDYHRQYEWV

SEQ ID NO: 12 - protein, 171 aa, Myxococcus xanthus
MNTPSLTNWPARLGYLLAVGGAWFAADQVTKQMARDGAKRPVAVFDSWWHFHYVENRAGAFGLFSSFGEEWRMPFFYVVGAICIVLLIGYYFYTPPTMKLQRWSLATMIGGALGNYVDRVRLRYVVDFVSWHVGDRFYWPSFNIADTAVVVGAALMILESFREPRQQLSPG

SEQ ID NO: 13 - protein, 475 aa, Myxococcus xanthus
MGTSEPVEPDHALSKPPPVAPVGAQALPRGPAMPGIAQLMMLFLRPTEFLDRCAARYGDTFTLKIPGTPPFIQTSDPALIEVIFKGDPDLFLGGKANNGLKPVVGENSLLVLDGKRHRRDRKLIMPTFLGERMHAYGSVIRDIVNAALDRWPVGKPFAVHEETQQIMLEVILRVIFGLEDARTIAQFRHHVHQVLKLALFLFPNGEGKPAAEGFARAVGKAFPSLDVFASLKAIDDIIYQEIQDRRSQDISGRQDVLSLMMQSHYDDGSVMTPQELRDELMTLLMAGHETSATIAAWCVYHLCRHPDAMGKLREEIAAHTVDGVLPLAKINELKFLDAVVKETMRITPVFSLVARVLKEPQTIGGTTYPANVVLSPNIYGTHHRADLWGDPKVFRPERFLEERVNPFHYFPFGGGIRKCIGTSFAYYEMKIFVSETVRRMRFDTRPGYHAKVVRRSNTLAPSQGVPIIVESRLPS

SEQ ID NO: 14 - protein, 318 aa, Myxococcus xanthus
MVDSVSKQARRKVFLFSGQGTQSYFMAKELFDTQTGFKRQLLELDEQFKQRLGHSILERIYDARAARLDPLDDVLVSFPAIFMIEHALARLLIDRGIQPDAVVGASMGEVAAAAIAGAISVDAAVALVAAQAQLFARTAPRGGMLAVLHELEACRGFTSVARDGEVAAINYPSNFVLAADEAGLGRIQQELSQRSVAFHRLPVRYPFHSSHLDPLREEYRSRVRADSLTWPRIPMYSCTTANRVHDLRSDHFWNVVRAPIQLYDTVLQLEGQGGCDFIDVGPAASFATIIKRILARDSTSRLFPLLSPSPASTGSSMG

SEQ ID NO: 15 - protein, 330 aa, Myxococcus xanthus
MTEAPAPRAPAQVPPPPSSPWALHTRGAASAPVNARKAALFPGQGSQERGMGAALFDEFPDLTDIADAILGYSIKRLCLEDPGKELAQTQFTQPALYVVNALSYLKRLREGAEQPAFVAGHSLGEYNALLVAGAFDFETGLRLVKRRGELMSGASGGTMAAVVGCDAVAVEQVLRDRQLTSLDIANINSPDQIVVSGPAQDIERARQCFVDRGARYVPLNVRAPFHSRYMQPAASEFERFLSQFQYAPLRCVVISNVTGRPYAHDNVVQGLALQLRSPVQWTATVRYLLEQGVEDFEELGPGRVLTRLITANKRGAPAPATAAPAKWANA

SEQ ID NO: 16 - protein, 417 aa, Myxococcus xanthus
MSTSPVQELVVSGFGVTSAIGQGAASFTSALLEGAARFRVMERPGRQHQANGQTTAHLGAEIASLAVPEGVTPQLWRSATFSGQAALVTVHEAWNAARLQAVPGHRIGLVVGGTNVQQRDLVLMQDAYRERVPFLRAAYGSTFMDTDLVGLCTQQFAIHGMSFTVGGASASGLLAVIQAAEAVLSRKVDVCIAVGALMDVSYWECQGLRAMGAMGTDRFAREPERACRPFDRESDGFIFGEACGAVVVESAEHARRRGVTPRGILSGWAMQLDASRGPLSSIERESQVIGAALRHADLAPERVDYVNPHGSGSRQGDAIELGALKACGLTHARVNTTKSITGHGLSSAGAVGLIATLVQLEQGRLHPSLNLVDPIDSSFRWVGATAEAQSLQNALVLAYGFGGINTAVAVRRSATES

SEQ ID NO: 17 - protein, 262 aa, Myxococcus xanthus
MQAASPPHRDYQTLRVRFEAQTCFLQLHRPDADNTISRTLIDECQQVLTLCEEHATTVVLEGLPHVFCMGADFRAIHDRVDDGRREQGNAEQLYRLWLQLATGPYVTVAHVQGKANAGGLGFVSACDIVLAKAEVQFSLSELLFGLFPACVMPFLARRIGIQRAHYLTLMTRPIDAAQALSWGLADAVDADSEKLLRLHLRRLRCLSKPAVTQYKKYASELGGQLLAAMPRAISANEAMFSDRATLEAIHRYVETGRLPWES

SEQ ID NO: 18 - protein, 256 aa, Myxococcus xanthus
MGIMTEGTPMAPVVTLHEVEEGVAQITLVDRENKNMFSEQLVRELITVFGKVNGNERYRAVVLTGYDTYFALGGTKAGLLSICDGIGSFNVTNFYSLALECDIPVISAMQGHGVGGGFAMGLFADFVVLSRESVYTTNFMRYGFTPGMGATYIVPKRLGYSLGHELLLNARNYRGADLEKRGVPFPVLPRKEVLPHAYEIARDLAAKPRLSLVTLKRHLVRDIRRELPDVIERELEMHGITFHHDDVRRRIEQLFL

SEQ ID NO: 19 - protein, 424 aa, Myxococcus xanthus
MLNLINNHAHGYVVTPVVLACNDAGLFELLRQGPKDFDRLAEALRANRGHLRVAMRMFESLGWVRRDADDVYAVTAAAAAHRSFPREAQSLFALPMDRYLRGEDGLSLAPWFERSRASWDTDDTLVRELLDGAIITPLMLALEQRGGLKEARRLSDLWSGGDGRDTCVPEAVQHELAGFFSAQKWTREDAVDAELTPKGAFIFERALLFAIVGSYRPMLASMPQLLFGDCDQVFGRDEAGHELHLDRTLNVIGSGHQHRKYFAELEKLIITVFDAENLSAQPRYIADMGCGDGTLLKRVYETVLRHTRRGRALDRFPLTLIAADFNEKALEAAGRTLAGLEHVALRADVARPDRLIEDLRARGLAEPENTLHIRSFLDHDRPYQPPADRAGLHARIPFDSVFVGKAGQEVVPAEVFHSLVEHLE

SEQ ID NO: 20 - DNA, 19053 bp, Myxococcus xanthus
gtcgacgttgacgtcgcccggtggcgtgccgtgtgtcttcttcgacgcggaggtgcgcgaggtggcggcggacggccggcgcgggccgctgttgtcgcgtgagcgcgcgtatgcgccggtactggcgctgcgtggccagcgcctccatgcttcggtgtccttttcgcccgcgtcgctgatggctccggtggaggtgcgccggtgcaaggccctgccaggcacggtgcccgcgtcctggtatcagacggcgcacccggaggccctgtcctgggagcgcgtgggcgcggtgggcgaatcctgcctcgtggtgggtgaactccggaggggccctgtcgagggcagctacgccctggtcggtcgggagggcggccccgcgatgttggtgctgggaccccaggctccggccacctgtgggacgctggcgcgccgggcctggcggcacttcgcggcggccggggtgctgtccatggccgcggccgtcgtcctgtcaggggcgctgtgagacgcgcggcgggggccgtaccgccgcgccagaaacgtgatgcgccgccaggcctcgcggtccgggcactgacgcccgggccgctcgggactcgctcaggcggctccggtgcttcgcgcggtggagaacacgagctgttcctcgctgtccgccacccgcacggtgagggtccgctccacgccggcgaggcccagcggcgtggactgcgccaggtccgagagcagggagcccgcagcgcgcaggtggaagccggtggtccacatgccctccagctcgccgaacacggtgcgcagctcgtccggggccttgcgttcgtcgatggcgcggcgcaggcgcaccatgccgctcacgaagttcctgcaccgcatgagcgtgttggggttcttggagatgacctccgcgaagctgaagttgccgccacccgggcgccactcgctttcgatgagctgcaggtccttgccaaggtcgcgcaccgtggggtccttctggaagtaccacttggcgatctgcgcggcgcggctgggtgacaagtcattcagcatgaggctgccttgctcctgcacctcgcggaggtaggcctcccagatgggggcgtagaccgcgcgccggaactcggcggaggcgcggggaagctgctcgctccagggcagcgcgcgggccagggcgcgtgagaagcgggacagctcgagcgtctggatgcggggcaccagggcgcggaacgagtcatccgccatcttcagctcgagccgcgtttcgatggggtgcttgcccatgtcctcctgcatggtgctgatgacggtggccgcgtccttcgcgtccagcacgccccagacgacggcgtcatccaccgcctgctgcaggtccttgcgcgacagcttcttctgccgtccccacagcatcaggtgcaggccgtagccgaagtcggaggccacgggggtgggagagaagcgcgtcatgtccgccttcaggtccaccacccactggccgcgggtgcccagcagcttgtcctcgtagccccggcgtccgaggaacgccatgcgccgggcgccctggaagttctcctgcgtcacccaggactgcttgctgacgttcttcgccttgagcagctcgaacgagcccagccccagtgagaagccgatggcctgctggcgcgtgagcgtggtggcgtgcaggtagttgcgcagctcgaactcgctctgctgggcggggaggctcttcatccacttcagcagctccaccaggttgcccttgagcagggactcgtggaagcgcatcgcggtgacgtcctccacgacgacctccagcagcgtggaggtctccgacaggcgcaggtattcgtactcgaagccctcggcgacctgcgtgcggacggcgttctccagcgactctgcgacgcgggccttgaagtcggcccaggcctgcggaaggttggccgggtccgcaagctgcgggtcgatgccaaggcgctccagcaccaggcccagcaccttcttctgattgtcgtccagcttcgccttgctggccttgtccaccaggccgtccatgatttccaccgcggtggtgcccttcttgacgaggtcgcgaaggacgggccccagcagcgcttccagcaactggcccagttgggccttcaccgtcgccgggtccagcagctccacggtgatgcccaggccggcggagagcgcctgctcatgggacttcaccttgcgcacggcgacctggatgcggccggcacggggacgggagaagctgagctggtagtcgtcggtgagggacacccgggcggacaggcccgccttggcgctggtcttcaacgcgagcagctcgttgccgcgcaggaagcccaggcggttgaggttggtggggaagatgtccgcccagttgagctccagccgtgtgtggagcgcgccgcggacatgccacgccaccgcgtcccccgtggtcagcttggccaggtcggtggcctgcatcagccgcagctcggacaggtcggagcgcacggcctcacgcatgttctggcccagcggatgcgcgcggtagtcgctgagcgtgacggacagctccgtctggttctcggaggcgaggaaggacaggctggcgctcacggcggccttcacgcgcgcggacacctggtagcgcatccacccgatgtcactgcccagcagcagttggggccggggccctggctcggcgctctccgggctcggctcgccgatgatgccgttttcgtccacgtcgcccagcgagttgaagttccggatggacgcttcgccggtggcttcgaaggagagggtgaagccgtgcatggcgagcttgggcggaaggattttctcttccgtgaaggtcccctgggccagcggcaccttgatgtcctggtcctgcagcttcacgtcgggcaccttgcccgccacgacgtcgggaagcttctccagcagcttgttgaccactttcatgcgcgtccccctgggctgaagcctcctgcacgtgggccggaggtctcttcgtcgtacgccgttgcccagctcggaacaaggcggataccagaaaagaccggtggtcagcggacagatgccctggagggtggggtgggagccgcccccgcgcggtgcgtcagggctcgtcgcccaatccgtactccttgagtttccgcgcgagcgtgttgcggccaatcgccagcacgcgggccgcggcggtgcggttgcccttcacggcgtccagcacgcgcaggatgtggcggcgttcgacctccgccagtggcagcaggcccgcatccaggggcgcaggcgcagtcggcgcgttgacaccctgagcggctgcgggaggcggcagggcggcccggtccacatcgggcaggggcaggtgctcttcgagaatctccccttcacagagcaccacggcgctctcgatacagttctccagctcccgcacgtttccgggccagcggtagcgcttgaggcgctccaccgcggcggcgctgaggcggggcggcgtcagccggtgcctccgggcgacggcggcgacgaagtggcgggcgagccgctcgatgtcctccgcgccgcgctcccgcagcggcggcagcaccacctcgaccaccttgatgcggtagtagaggtcctcgcggaagcggccctcggccaccatgcgggccaggtcccgatgggtggccgcgacgatgcgcacgtccaccttcacggcctgggtgcctcccacgcgctcgaactcgcgatcctggatgacccgcagcaacttgccctgcaccggcaggggcagctcgccaatctcgtcgatgaacacggtgccgccgctggccgcttcgaacttgccgggcacgcggtggtccgcgccggtgaaggcgccgcgttcgtggccgaagagctcgttctcgatgagcgtggcgggcagcgccgcgcagtccaccttgatgaagggctggtccctgcggggaccattcacgtggacggcacgggcgaacagctccttgccgctgccactctcgccgcgcagcagcaccgtcgcatcggtgggcgcggccttgcgcaccagtcggtagatggcctggagctgcggggactcgccgatgatgcggttgaagaagtagcccaccggtacctggggctgctccttcgcgcgctggagctcttgatagaggctggtgctctggagggcggtgctcacctgcgaggcgatggcggtgagccgctgcgtgtcctcgtcggtgaagcggtcctcgccgcggcggttgaggacctggagcacgccgtagagggcgccgtccccgtcgcgcagtggcacggcgagcaggctggtggtgcggtagcccgtcatccggtcgatgtccgcgaagaagcgctgctcgccgcgcgggtccggcacgttgatggcgtgccccgccttggcgacggtgccggcgacgccctggcccagcttgacgcgaatctgggacacctcgggcaggtgcgcggcgcggctgaacagctcgcggcgggccgggtccagcagccagagcgtgccgcggtccgcttgcagggtgatggcgatgcggtccatcagcgtctggaggaacgcgtcgaggtccacctccctgccgacgagtcctccgaaggggaggaggacctgggagacgtccgagggggcttggggcatggcgggcaacggcggcaggacgaaggcggaggccgcaccataacatccagagggcatgggactgccccctctcaggccgcgcggcccagcaccagccgctggccttcgcgtgcgggctcgcacacggggaagagggcgcgggcctgctccgccatgtcctcgagcatgtcgtcgccgtgcgccgggtcatggtggaacaggcacagccggcgcgcccccaccagcccggccacgcccgcggcatccatcatggtggagtggccccagcccttcttcgccacgcccttgcggccctcgtattcgtccggcgtgtactgcgcatccaggcacaggacgtccgcgccctcgaagaggcggcccacctccggcgcgagctcctgcacccgcacctccacgtccgtggcgtagacgaacgaatggccatccgcctccaggcggtacgccaggcacccctgcgggtgcggcacgtcgatgggcgtgacgcggaaggggcccacctccacgggtcgggcatgcaacgccgagcggaagtccatccgcgagcgcatggtgctcagcggcaccggaaaatgaagcggctgcatctgcgcggccaactcggactggagcgcctgggccccattcgcgcccggaccgtagagcgtcagctcggacgtgggcagccaggccggcgtgaagaaggggaagccctgcacgtggtcccaatgcagatgcgagaagaagagcgtggcctcctggggcgcgccctcgcgcatcatgatttcgcccagtgcgcggatgcccgtccccgcatccaggatgaggcggtggccctggctggtcacctccacgcaggccgtgttgccaccaatgcgcgagcccgacaccgcgatgctcccccgaacgccatgaaaccggacttccatcgtaagtctccttgaatggggggcctccgcctgggacgccctcatgcccggagcctcagagcacggggtgtgccattcccaaatgcccggaatcaggagcgcgggcctcgggctcgtccaccggtgctccagaatggatcgcgctcgcctggtgcgggcgatccaaagcggtgcaggtcgcccgcaggacggggcggcgggcacgtcttccaacgtcccacggcagtcctgtcttcagatctctcccgatgcgggaaggcgtccaggaggttgcacccggcatcgagcggggctgtgtgtttcaagtcttgtcggagcctcggacacaaccgtctgggttctgggaatgcgccggcttccgttcactccagagtgattcaatggctctcgagtgcaggtttagcaatcctcgggccgtaaccacgccgttgaaggcagtcacgctctcgtcacgcttggggtgtttccagcttcaacggtgtttatccttcagggcggtttgcttgacacgctgcctcatggaagcgtatgcaaaacaatgaaaacggtgtcgttgccgagccttagggcctccagaacgccatcctcgcggacccaggcagccggaatttgagacggggctgtcagcggtttgaacgcaaggatgcggcgggggttgtggcggcagcccgaccagaattcggttggtgtgccagttattgtcagattctgagaaatagcaggctggggggaagttgcaatgcctgggccgcggtgtgctgagaacgattgggttgcattgctcgtccgcgtcaatcacgagaaagtggctgccgctcagttggggaaacacggctacgagttcttcctgccgacgtacacgcctcccaagtcctcgggtgtgaaggcgaagcttccgctcttccccgggtaccttttctgtcgttaccagccgctcaatccgtaccgcatcgtccgggcgcccggggtcatccggctgctcggaggtgacgcggggccggaagccgtgcccgcacaggaattggaggccatccgccgggtcgcggattcgggtgtctcttccaatccctgtgactatctgcgggtggggcagcgcgtgcgcatcatcgaagggcccctgacaggtctggaaggaagtctggtgacgagcaagagccaactccggttcattgtctccgtggggctgctacagcgctcggtgtccgtggaggtgagcgccgagcaactggaaccgatcaccgactgattccgcggacatccccttccattccttcatcaccccgacccgcagcaaggcttcagggaccgtgagtcgttccatggacaagagaattattttcgacatcgtcaccagcagtgttcgggaggtggtacccgaactcgaatcacatccgttcgagccggaggatgacctggtcggactgggcgcgaactcgctcgaccgcgccgaaatcgtcaacctcacgctggagaagctggcgctcaacatcccccgggtcgagctgattgacgcgaagaccattggcgggctggtggacgtccttcacgcgaggctgtgaggcgaagccatggggccggtcgggattgaagccatgaatgcctactgtggcatcgccaggttggatgtgttgcagctggcgacccaccgtggcctggacacctcccgcttcgcgaacctgctcatggaggagaagaccgtcccgctcccctatgaggaccctgtcacctacggcgtgaatgccgcccggcccatcctggaccagttgaccgcggcggaacgggacagcatcgagctgctggtggcttgcacggagtcctcgttcgacttcggcaaggccatgagcacctacctgcaccagcacctggggctgagccgcaactgccggctcatcgagctcaagagcgcctgctactccggggtcgccgggctgcagatggccgtcaacttcatcctgtccggcgtgtcgccgggggccaaggccctggtggtggcctccgacctgtcgcgcttctccatcgccgaagggggagatgcctccacggaggactggtccttcgcggagccgagctcgggtgcgggcgcggtggccatgctggtgagcgacacgccccgggtgttccgcgtcgacgtgggggcgaacggctactacggctacgaggtgatggatacctgccgcccggtggcggacagcgaagcgggagacgcggacctgtcgctcctctcgtacctggactgctgtgagaacgccttccgggagtacacccgccgcgtccccgcggcgaactacgcggagagcttcggctacctcgccttccacacgccgtttggcggcatggtgaagggcgcccaccgcacgatgatgcgcaagttctccggcaagaaccgcggggacatcgaagcggacttccagcggcgagtggcccccgggctgacctactgccagcgcgtggggaacatcatgggcgcgacgatggcgctctcgctcctcgggaccatcgaccacggcgacttcgccaccgcgaagcggattggctgcttctcgtatggctcggggtgcagctcggagttcttcagcggcgtggtgacggaggaagggcagcagcggcagcgcgccctggggctgggagaagcgctggggcgccggcagcagctctccatgccggattacgacgcgctgctgaaggggaacggcctggtgcgcttcgggacccggaacgccgagctggatttcggtgtcgtcggcagcatccggccgggcgggtggggcaggcccttgctcttcttgtcggcgattcgtgacttccatcgcgactaccaatggatttcctagcctcggggcttcgagcaaagccatgtccagcgtagcgacggccgtccccctgacggcccgtgacagcgcggtgagccgccggctgcgaatcacccccagcatgtgcggccagacgtccttgttcgccgggcagattggcgactgggcatgggacaccgtcagccgcctgtgtggcacggacgtgctgaccgcgaccaacgcctcaggcgcgcccacctacctggccttctattacttccgcatccggggcacgcccgcgctgcatcccggcgcgctgcgcttcggcgacacgctggacgtcacgtcgaaggcgtacaacttcggcagcgaatccgtcctgacggtgcaccgcatctgcaagacggcggagggcggcgctccggaggcggatgccttcggccatgaagagctgtacgagcagccccagccaggccgcatctacgcggagaccttcaaccggtggatcacgcgctcggacggcaagtcgaacgagagcctgatcaagtcctcgcccgtggggttccagtacgcacacctgccgctcttgccggacgaatactcgccgcggcgggcctatggggacgcgcgggcgcggggcacctttcacgatgtggactccgcggagtaccggctgaccgtggaccgcttcccgctgcgctacgcggtggacgtcatccgggacgtcaatggggtggggctcatctacttcgcgtcgtatttctcgatggtggactgggccatctggcagctggcgaggcaccagggacgcagcgagcaggccttcctgtcgcgcgtggtgctggaccagcaactgtgcttcctcggcaacgcggcgctggacaccaccttcgacatcgacgtgcagcactgggagcgggtgggcggcggggaagagctgttcaacgtgaagatgcgcgagggcgcgcagggccgggacatcgccgtggcgacggtcaaggtgcgcttcgacgccgcttcggaaggaggccgccgtgggtgagccgatgacagacgaacaaatccgcggagtcgtgcaccagtccatcgtgcgcgtcctgccccgcgtgcgctccaacgagattgcgggccacttgaacctccgcgagctgggcgcggactccgtggaccgggtcgagattctcacgtccatcctggacagcctgcggctgcagaagacgccactggcgaagttcgccgacatccgcaacatcgacgcgctggtggcgttcctggccggtgaggtcgcgggtggctgagcgggttcccggcggagtcggcatcgaggccatcaacgcctacggcggcgccgcctccattccggtgttggacttgttccggggccggcggctggaccccgaagcgattctccaacctgatgatgcaggagcgcggcgtcgcgctgccgttcgaggaccccgtcaccaacgcggtcaatgcggcgcggcccatcctggacgcgatgtcgcccgaggcccgggagcgcatcgagctcctggtcacctcgagcgagtccggcgtggacttcagcaagtccatctcctcgtatgcgcacgagcacctggggctgagccgccactgccggttcctggaggtgaagcaggcgtgttacgccgccaccggagcgctccagctagcgctgggctacatcgcgtcgggcgtgtcaccgggggccaaggccctggtgattgccacggacgtgacgctggtggacgagagcggtctgtactccgagccggcgatgggcaccggcggcgtcgccgtgctgctgggcgacgagccgcgcgtgatgaagatggacctgggagcgttcggcaactacagctacgacgtcttcgacaccgcgcggccctcgccggagattgatatcggcgacgtggaccggtcgctcttcacgtacctggactgcctcaagcacagcttcgccgcgtatggccgccgggtggacggtgtcgacttcgtgtcgacgttcgactacctggcgatgcacacgccgttcgccggactggtgaaggccgggcaccgcaagatgatgcgcgagctcaccccgtgcgacgtggacgaaatcgaagcggacttcggccggcgcgtgaagccgtcactgcagtacccgagtctggtcgggaacctgtgctccggctccgtgtacctgagcctgtgcagcatcatcgacaccatcaagcccgagcggtccgctcgggtgggaatgttctcctatgggtcgggttgctcgtcggagttcttcagcggcgtcatcggcccggagtccgtgtccgcgctagctgggttggacatcggtggccacctccgggggcgccgccagctcacgttcgaccaatatgtcgaattgctgaaagagaaccttcgctgtctggttccaacgaagaaccgggacgtggacgtggagcgctacctcccgctggtgacgcggacggcgagccgcccgcgcatgctcgccttgcgaagggtcgtggactatcatcgtcagtacgagtgggtgtagctcatacgccacctccaattccgacgaatgaacactccttccttgacgaactggcctgcccgcctgggctatctccttgccgttggcggcgcatggttcgcggccgatcaagtcaccaaacagatggcgcgcgacggggcgaaaaggcccgtcgcggtcttcgatagctggtggcacttccactacgtggagaaccgagcgggtgcgttcggtctgttctccagcttcggcgaagagtggcgcatgcctttcttctacgtcgtgggcgccatctgcatcgtgttgctgattggctactacttctacacgccgccgacgatgaagctccagcgctggtcgctggcgacgatgattggcggcgcgttgggcaactacgtggaccgggtgcgcctgcgctacgtggtggatttcgtgtcatggcacgtgggggaccgcttctattggccctccttcaacatcgcggacacagcggtagtcgtaggggccgccctgatgatcctggagtcgttccgcgagccgcgtcagcagttgtctcccggataggccccgccatgggtgtgcggtcggccgccgggccaaggactggagttcatggggacctcagagccagttgagccggaccacgccttgtcaaaaccaccgcctgtcgcgcccgtcggcgcccaggcactgcctcgcggtccggcaatgcccggcatcgcgcagttgatgatgttgttcctgcggcccacggagttcctggaccgctgcgccgcccggtacggtgacaccttcaccctcaagattccggggacgccgccgttcatccagaccagcgatcccgccttgatcgaggtcatcttcaagggtgacccggacctcttcctcggagggaaggcgaacaacgggttgaagccggtggtgggtgagaactcgctgctggtgttggacgggaagcggcaccggcgtgatcgcaagctcatcatgcccaccttcctgggtgaacggatgcatgcgtatggctcggtcatccgggacatcgtcaatgcggcgcttgaccggtggcccgtcgggaagccgttcgcggtccatgaagagacgcagcagatcatgctggaggtgattctccgggtgattttcggcctggaggacgcccggaccattgcccagttccggcaccacgtgcaccaggtgctcaagctggccctgttcctgttcccgaacggggagggcaagcccgccgccgagggcttcgcgcgggccgtgggcaaggcgtttccctccctggacgtgttcgcgtcgctgaaggcgattgacgacatcatctaccaggagattcaggaccgccggagccaggacatcagcgggcggcaggacgtgctctcgctgatgatgcagtcgcactacgacgacggctccgtgatgacgccccaggagctgcgcgacgagctgatgacgctgctgatggcgggccacgagacgagcgcgaccatcgccgcgtggtgcgtctaccacctctgccgtcacccggatgcgatgggcaagctgcgtgaggagatcgcggcccacacggtggacggcgtgctgccgctggcgaagatcaacgagctgaagttcctggatgccgtggtcaaggagacgatgcgcatcacgcccgtcttcagcctggtggctcgcgtgctcaaggagccacagaccattggcggaacgacgtacccggcgaacgtggtgctgtcgcccaacatctacggcacgcaccatcgcgcggacctgtggggagacccgaaggtctttcggccagagcgtttcctggaggagcgggtgaatccgttccactacttccccttcggagggggcatccggaagtgcatcgggacgagcttcgcctactacgagatgaagatcttcgtctcggagacggtgcgccgcatgcgcttcgataccaggcccggctaccacgcgaaggtggtgcgccggagcaacacgctggcgccgtctcagggcgtgcccatcatcgtcgagtcgcggctgccgagctgaaccgcttggccccaccatctccagcgcggtgaacatcatggtcgattcagtgtcgaaacaggcacggcggaaggtgtttcttttttccggccagggcacccagtcgtacttcatggccaaggagctgtttgacacccagacggggttcaagcggcagctgctggagctggacgagcaattcaagcagcggctggggcactcgattctcgagcgaatctatgacgcgcgcgccgcgcggttggatccgctcgacgatgtcctggtgtcctttcccgccatcttcatgattgagcatgcgctggcgcggctgctcatcgaccggggtatccagccggacgctgtcgtgggcgccagcatgggcgaggtggcggcggcggcgattgcgggcgcaatctcagtggacgcggccgtggccctggtggcggcgcaggcccagctctttgcccgtacggcgccgcggggcggcatgctcgcggtgcttcacgaactggaagcctgccggggcttcacgtccgtcgcgcgggatggcgaggttgcagccatcaactacccgtcgaacttcgtccttgcggcggatgaggcgggcctgggacggattcagcaggaactctcccaacgctcggtggcgttccaccggttgccggtgcgctacccctttcattcctcgcacctggacccgctgagggaggagtaccgaagccgcgtccgcgcggattcgctgacgtggccgcgaatccccatgtactcgtgcaccaccgcgaaccgggtgcacgacctgcgcagcgaccacttctggaacgtggtccgcgcgcccatccagctgtacgacaccgtcctgcaactggaggggcagggcggctgcgacttcatcgacgtcggccccgccgcgtccttcgcgaccatcatcaagcgcatcctcgcgcgggactccacgtcacggctcttcccgttgctcagcccttctcccgcatcgaccgggagctcgatggggtgacgcggagctgcgcgatgacggaggcgcccgcacccagggcgcctgcgcaggtgccgccgccgccgagctcgccctgggcgctgcacacccgaggagcggcgagcgcgccggtgaatgcccgcaaggccgcgctcttcccggggcagggctcgcaggagcgcggcatgggggccgcgctcttcgacgagttcccggacctgacggacatcgccgacgccatcctggggtattccatcaagcgtctctgtttggaggacccaggcaaggagctggcgcagacgcagttcacccagccggcgttgtacgtggtgaacgcgctcagctacctgaagcggctgcgtgaaggagcggagcagccggccttcgtcgcgggccacagcctgggcgagtacaacgcgctgctggtcgcgggggccttcgacttcgagacgggactgcggctggtgaagcggcggggcgaactcatgagcggcgcgtccggagggaccatggccgcggtggtgggctgtgatgccgtggccgtggaacaggtccttcgagaccgtcagctgaccagtctggatatcgccaacatcaactcgcccgaccagattgtggtctccggaccggcgcaggacatcgagcgggcacggcagtgtttcgtggaccgtggcgcgcggtacgttccgctcaacgtgcgagcgccgtttcactcgcgctacatgcagccggccgccagcgagttcgagcgcttcctgtctcagttccagtacgcgccgctccggtgcgtggtcatctccaacgtcacgggccgaccttacgctcatgacaacgtggtgcaggggctggctctgcaactgcgcagcccggtgcagtggacggccaccgtccgctacctcctggaacagggcgtggaggacttcgaggagctgggccccggccgcgtgctgacccgcctcatcaccgcgaacaagcggggcgcccccgcaccggccaccgccgcgcccgcgaagtgggcgaatgcctgagccctccggagcgtcgttgaaatcctcggccggtgggccgtccggctgctgagaccactgaatgtccacctcacctgtgcaggagctggttgtctcggggttcggggtcacctccgccattggccagggggccgcgtccttcacctcggcgctgctggagggcgcggcacggttccgggtgatggagcggccgggccgtcagcatcaggccaacgggcagacgacggcccacctgggggcggaaatcgcctcgctggccgtgcccgaaggcgtcaccccacaactgtggcgctcggccacgttttcggggcaggccgcactggtgaccgtccacgaggcctggaacgcggcgcgcctccaggccgtccccggacaccggattggattggtggtggggggcaccaacgtgcagcagcgcgacctggtgctgatgcaagacgcctatcgcgagcgggtgccctttctgcgggcggcctacgggtcgaccttcatggacaccgacctcgtgggcctctgcacgcagcagttcgccatccacgggatgtccttcacggtgggaggcgcatcggccagtggcctgctggcggtcatccaggccgcggaggcggtgctctcaagaagggtggacgtttgcatcgccgtgggggcgctgatggacgtctcctactgggaatgccagggcctgcgggccatgggcgcgatgggcaccgaccggttcgcgcgggagccggagcgtgcctgccggcccttcgaccgggagagtgatggcttcatctttggagaggcgtgcggcgccgtggtggttgagtctgcggagcacgctcggcgacgcggggtgactcctcgcggcatcctgtcgggctgggccatgcagttggacgcgagccgcggcccgttgtcgtccatcgaaggggagtcgcaggtgattggggctgcgctgcggcacgcggacctcgcgccggagcgggtggactacgtgaatcctcacggcagcggttcgcgtcagggggatgccatcgagctgggggccttgaaggcgtgcggcctgacgcacgcccgggtcaacaccacgaagtccatcaccgggcatggcctgtcctcggcgggtgccgtggggctcatcgccacgctggtccagttggagcagggccggctgcacccgtccttgaacctggtggacccgattgattcatcgttccgctgggtgggggccaccgcggaggcccagtccctccagaacgcgctggtgctcgcctacggcttcggcggcatcaacaccgctgtcgccgtgcgccggagcgccacggagagctgacacgcccatgcaagccgcttcccctccgcaccgcgactaccagacgctccgggtccgcttcgaggcgcagacctgttttctccagctccaccggccggatgcggacaacaccatcagccgcacgctgattgacgagtgccagcaggtgctcacgttatgtgaggagcacgccaccacggtggtgctcgaaggcctgccacacgtgttctgcatgggcgcggattttcgagccatccacgaccgggtcgacgacggccgccgggagcaaggcaacgcggagcagctgtaccggctgtggctgcaactggcgacaggcccctacgtgacggtcgcccatgtgcagggcaaggccaacgcgggcggcctgggcttcgtcgccgcgtgcgacatcgtgctggcaaaggcggaggtccagttcagtctctccgagctgctgttcgggctgttccccgcctgcgtgatgccgttcctcgcccggcgaatcggcatccagcgggcgcactacctgacgctgatgacgcggcccatcgacgcggcccaggcgctgagctgggggttggcggacgcggtggacgccgatagcgagaagctgttgcggctccacttgcgcaggctgcggtgcctgtcgaagccagcggtgacccagtacaagaagtacgcctccgagctgggcggccagctgctcgcggccatgccccgggccatctccgccaatgaggcgatgttctccgaccgcgccacgctggaagccatccatcgctacgtggagacaggccgactcccatgggaatcatgacggaaggaacgccaatggcgccggtggtcacgctccatgaggtggaggagggggtggcgcagatcaccctggtggatcgcgagaacaagaacatgttcagcgagcagctcgtgcgcgagctcatcaccgtgttcggcaaggtgaatggaaacgagcgctaccgcgcggtggtgctcaccggctacgacacctacttcgcgctcggcgggaccaaggccggcctgctgtccatctgcgacggcatcggctccttcaacgtcaccaacttctacagcctcgcgctggagtgcgacatcccggtgatttccgccatgcagggacatggcgtaggcggcgggttcgcgatggggctgttcgcggacttcgtggtcctgagccgggagagcgtctacacgacgaacttcatgcgctacggcttcacgccggggatgggcgccacgtacatcgtgccgaagcggctggggtactcgctcgggcatgagctcctgctcaacgccaggaactaccgcggcgccgacctggagaagcggggcgtgccttttccggtgttgccgcgcaaggaagtcttgccccacgcctacgagattgcgagggacctggccgcgaaacctcggctgtcgctcgtgacgctcaagcggcacctggttcgcgacatccgccgagagcttccggacgtcatcgagcgtgagctggagatgcacggcatcaccttccatcacgacgacgtgaggaggcgcatcgagcagctgttcctctgaggcgcgcccctatgttgaacctgatcaacaaccacgcacacggttatgtggtcacgcccgtggtcctggcctgcaacgacgctggcctgttcgaactcctgcggcagggaccgaaggacttcgaccggttggcggaggcattgcgtgccaaccggggacatctgcgcgtcgcgatgaggatgttcgaatcgctcggctgggttcgccgcgacgcggatgacgtgtacgcggtgacggcggcggcggccgcgcatcggtccttcccccgcgaggcgcagtcgctcttcgcgctgcccatggaccggtacctgcgcggggaggacggcctgtccctggcgccgtggttcgagcgctctcgggcgtcgtgggataccgatgacacgctggtgcgcgagctgctcgacggcgccatcatcacgccgctgatgctcgcgctggagcagcgtgggggcctcaaggaggcgaggcgtctgtccgacctgtggtccgggggggatggaagggacacgtgcgtccccgaggccgtccaacacgagctggccgggttcttctccgcgcagaagtggacgcgtgaggacgccgtcgacgcggagctcacgcccaagggcgccttcatcttcgagcgggcattgctcttcgccatcgtcggctcgtaccggccgatgctggccagcatgccgcagctgctcttcggtgactgcgaccaggtcttcgggcgggacgaagcgggccacgaactgcacctggaccgaaccctcaacgtgattgggagcggccaccagcaccggaagtacttcgcggagctggagaagctcatcatcaccgtcttcgatgccgagaacctgtcggcacagccgcgctacatcgcggacatggggtgcggtgacggcacgctcctgaagcgggtgtatgaaacggtgcttcggcacacgcggcggggaagggcgctcgaccggtttccgctcacgctcatcgccgcggacttcaacgagaaggcgctcgaagccgctgggcggacgctggccgggttggagcacgttgccttgcgcgcggacgtggcgcggccggaccgtctcatcgaggacctgcgggcgcgcgggctagccgagcctgagaatacgctgcacatccgctcgtttctcgaccacgaccgtccctaccagcctcccgcggacagggcggggctccacgcccggattccgttcgattcggtgttcgtgggcaaggcgggccaggaggtggttccggcggaggtgttccacagcctggtggagcacctcgag

(19) United States
(12) Patent Application Publication
(10) Pub. No.: US 2004/0235107 A1
(43) Pub. Date: Nov. 25, 2004

(54) Biosynthesis of TA antibiotic

(75) Inventors: Eugene Rosenberg, Givat Shmuel (IL); Eliora Ron, Tel Aviv (IL); Elisha Orr, Leicester (GB); Yossi Paitan, Rishon LeZion (IL)

Correspondence Address: MARTIN MOYNIHAN, c/o ANTHONY CASTORINA, SUITE 207, 2001 JEFFERSON DAVIS HIGHWAY, ARLINGTON, VA, 22202, US

(73) Assignee: Ramot At Tel Aviv University Ltd.

(21) Appl. No.: 10/848,111

(22) Filed: May 19, 2004

Related U.S. Application Data
(63) Continuation-in-part of application No. 09/710,262, filed on Nov. 10, 2000.
Application No. 09/710,262 is a continuation of application No. 09/240,537, filed on Jan. 29, 1999, now abandoned.

(51) Int. Cl. C07H 21/04

(52) U.S. Cl. 435/69.1; 435/252.33; 530/350; 536/23.5; 435/320.1

(57) ABSTRACT

A method of producing an antibiotic TA comprising (i) expressing in a host cell an exogenous polynucleotide sequence encoding at least one polypeptide selected from the group consisting of SEQ ID NOs: 1 and 3-19; and (ii) culturing the host cell under conditions suitable for synthesis of the antibiotic TA, thereby producing the antibiotic TA.

This is a continuation-in-part of U.S. patent application Ser. No. 09/710,262, filed Nov. 10, 2000, now allowed, which is a continuation of U.S. patent application Ser. No. 09/240,537, filed Jan. 29, 1999, now abandoned, the content of all of which is hereby incorporated by reference.

### FIELD AND BACKGROUND OF THE INVENTION

The present invention relates to methods of producing antibiotic TA, or derivatives thereof and, more particularly to methods of producing antibiotic TA or derivatives thereof by expressing a polynucleotide sequence encoding polypeptides participating in synthesis of the antibiotic in a host cell.

Polyketides constitute a large and highly diverse group of secondary metabolites synthesized by bacteria, fungi and plants, with a broad range of biological activities and medical applications. They include anti-cancer agents (Daunorubicin), antibiotics (tetracyclines, erythromycin etc.), immunosuppressants (macrolide FK506) and compounds with mycotoxic activity (aflatoxins, ochratoxins, ergochromes, patulin etc.). Polyketides are synthesized by repetitive condensations of acetate or propionate monomers in a similar way to that of fatty acid biosynthesis. Structural diversity of polyketides is achieved through different thioester primers, varying chain extension units used by the polyketide synthases (PKSs), and variations in the stereochemistry and the degree of reduction of intermediates. Diversity is also achieved by subsequent processing, such as alkylations, oxidations, O-methylations, glycosylations and cyclizations. Genetic studies indicated that gene organization of functional units and motif patterns of various PKSs are similar. This similarity was used to identify and obtain new PKS systems in both gram negative and gram positive bacteria.

PKS systems are classified into two types: type I PKSs are large, multifunctional enzymes, containing a separate site for each condensation or modification step. These represent “modular PKSs” in which the functional domains encoded by the DNA sequence are usually ordered parallel to the sequence of reactions carried out on the growing polyketide chain. Type II PKSs are systems made up of individual enzymes, in which each catalytic site is used repeatedly during the biosynthetic process.

The polyketide antibiotic Tel-Aviv (hereinafter TA; Rosenberg et al., 1973; Rosenberg et al., 1984) is an antibacterial antibiotic synthesized by the gram negative bacterium Myxococcus xanthus in a unique multi-step process incorporating a glycine molecule into the polyketide carbon chain, which is elongated through the condensation of 11 acetate molecules by a type I polyketide synthase (PKSs).

The antibiotic TA was crystallized and its chemical properties were determined. It is a macrocyclic polyketide synthesized through the incorporation of acetate, methionine, and glycine. It inhibits cell wall synthesis by interfering with the polymerization of the lipid-disaccharide-pentapeptide and its ability to adhere avidly to tissues and inorganic surfaces makes it potentially useful in a wide range of clinical applications, such as treating gingivitis.

The present invention provides novel methods of producing antibiotic TA, or derivatives thereof, by expressing in a host cell an exogenous polynucleotide sequence encoding one or more polypeptides participating in the synthesis of the antibiotic.

### SUMMARY OF THE INVENTION

According to one aspect of the present invention there is provided a method of producing an antibiotic TA including (i) expressing in a host cell an exogenous polynucleotide sequence encoding at least one polypeptide selected from the group consisting of SEQ ID NOs: 1 and 3-19; and (ii) culturing the host cell under conditions suitable for synthesis of the antibiotic TA, thereby producing the antibiotic TA.

According to another aspect of the present invention there is provided a method of producing a modified antibiotic TA, including (i) mutating a polynucleotide sequence encoding at least one polypeptide selected from the group consisting of SEQ ID NOs: 1 and 3-19; (ii) expressing a mutated polynucleotide sequence resulting from step (a) in a host cell; and (iii) culturing the host cell under conditions suitable for synthesis of antibiotic TA, thereby producing the modified antibiotic TA.

According to further features in preferred embodiments of the invention described below, the step of expressing is effected by transforming the host cell with a nucleic acid construct including the exogenous polynucleotide under the transcriptional regulation of a promoter functional in the host cell.

According to further features in the described preferred embodiments the nucleic acid construct further includes a nucleotide sequence encoding a signal for secretion of the at least one polypeptide to the outside of the host cell.

According to still further features in the described preferred embodiments the method of producing an antibiotic TA further comprising regulating an expression or activity of at least one endogenous polypeptide capable of modulating the synthesis of the antibiotic TA.

According to still further features in the described preferred embodiments the method of producing an antibiotic TA further comprising isolating the antibiotic TA produced in the host cell.

According to still further features in the described preferred embodiments the host cell is a eukaryotic or a prokaryotic host cell.

According to still further features in the described preferred embodiments the prokaryotic host cell is E. coli.

According to still further features in the described preferred embodiments the prokaryotic host cell is a Myxococcus species.

According to still further features in the described preferred embodiments the Myxococcus species is Myxococcus xanthus.

According to still further features in the described preferred embodiments the mutation is effected by a deletion of one or more nucleotides.

According to still further features in the described preferred embodiments the mutation is effected by an insertion of one or more nucleotides.

According to still further features in the described preferred embodiments the mutation is effected by a substitution of one or more nucleotides.

The present invention successfully addresses the shortcomings of the presently known configurations by providing a method of producing antibiotic TA, or derivatives thereof, by expressing polynucleotides encoding polypeptides participating in synthesis of TA antibiotic in a host cell.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1 illustrates a physical map of TA antibiotic gene cluster. A DNA fragment of about 8-10 kb derived from cosmid pPYCC64 and designated “Region 1” encodes the polypeptide “Tal” which is involved in the incorporation of the glycine into the TA polyketide chain. DNA fragment of about 20 kb derived from cosmid pPYCC44 and designated “Region 2” encodes the polypeptides TaA, TaB, TaC, TaD, TaE, TaF, TaG, TaH, TaI, TaJ, TaK, TaL, TaM, TaN, TaR3, TaR2 and TaR1, which are responsible for the regulation or the post-modification of TA antibiotic. Restriction enzymes: S, SalI; X, XhoI; Bm, BamHI; Ei, EcoRI; K, KpnI; H, HindIII; and Bg, BglII.

### DESCRIPTION OF THE PREFERRED EMBODIMENTS

The present invention is of a method of producing antibiotic TA and derivatives thereof. Specifically, the present invention is of methods which utilize a host cell capable of expressing an exogenous polynucleotide encoding one or more polypeptides participating in the synthesis of TA antibiotic to generate the TA antibiotic.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

U.S. Pat. No. 3,973,005 teaches methods of producing the TA antibiotic using organisms which normally synthesize this antibiotic; this reference does not describe or suggest genetically modified organism capable of producing this antibiotic product.

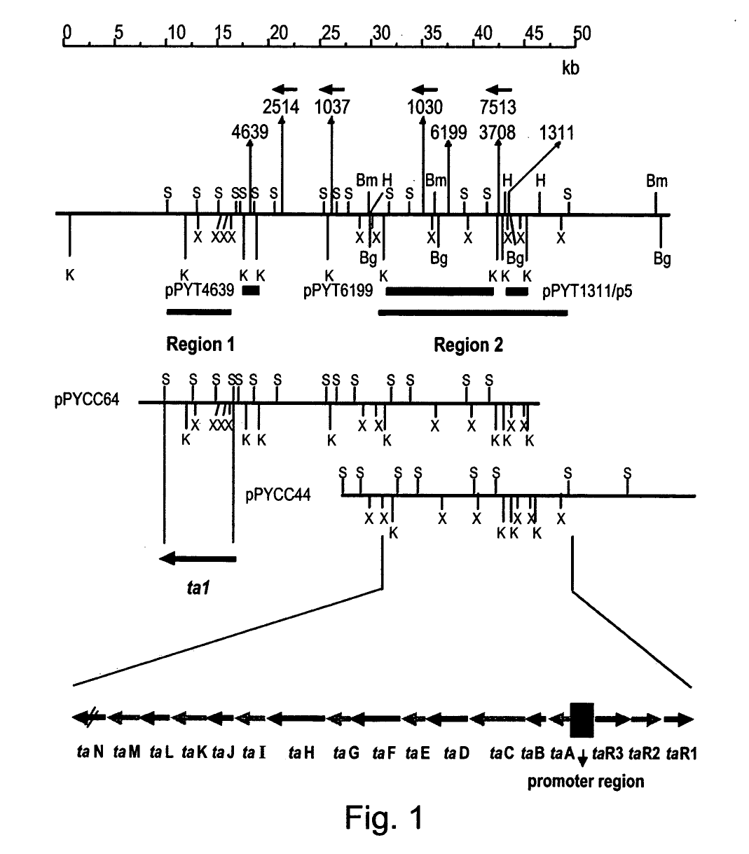

While reducing the present invention to practice the present inventors identified, cloned and characterized a DNA fragment of at least 42 kb encoding genes involved in antibiotic TA production in Myxococus xanthus (FIG. 1). This fragment includes a large region (designated Region 2) of about 20 kb, encoding the polypeptides designated TaA, TaB, TaC, TaD, TaE, TaF, TaG, TaH, Tal, TaJ, TaK, TaL, TaM, TaN, TaR3, TaR2 and TaR1, which are responsible for the regulation or the post-modification of TA. An additional fragment (designated Region 1) of approximately 8 kb is located 10-20 kb downstream of the post modification region, encoding the polypeptide designated Ta1. The polypeptide Ta1 is involved in the incorporation of the glycine into the TA polyketide chain. This novel polypeptide is made up of a peptide synthetase unit lying between two PKS modules (Example 1).

Thus, according to one aspect of the present invention, there is provided a method of producing antibiotic TA. The method includes expressing in host cells an exogenous polynucleotide sequence encoding one or more polypeptides selected from the group consisting of SEQ ID NOs: 1 and 3-19, followed by culturing the host cells under conditions suitable for the synthesis of the antibiotic.

As used herein the term “expressing” refers to the transcription and optionally translation of a polynucleotide sequence to produce an mRNA or a polypeptide product.

The host cell of the present invention can be any suitable prokaryotic or eukaryotic host cell. Preferably, the host cell is a member of the family Myxococcacceae which includes the genus Angiococcus (e.g., A. disciforrmis), the genus Myxococcus (e.g., M. stipitatus, M. fulvus, M. xanthus, M. virescens) and the genus Corallococcus (e.g., C. macrosporus, C. corralloides, and C. exiguus). More preferably, the host cell is a Myxococcus species, most preferably, the host cell is Myxococcus xanthus. Alternatively, the host cell can be an E. coli.

The exogenous polynucleotide of the present invention includes at least a portion of the DNA sequence set forth in SEQ ID NOs 2 (encoding Tal polypeptide, SEQ ID NO: 1) or 20 [encoding TaA (SEQ ID NO:6); TaB (SEQ ID NO:7); TaC (SEQ ID NO:8); TaD (SEQ ID NO:9); TaE (SEQ ID NO:10); TaF (SEQ ID NO:11); TaG (SEQ ID NO:12); TaH (SEQ ID NO:13); TaI (SEQ ID NO:14); TaJ (SEQ ID NO:15); TaK (SEQ ID NO:16); TaL (SEQ ID NO:17); TaM (SEQ ID NO:18); TaN (SEQ ID NO:19); TaR3(SEQ ID NO:5); TaR2 (SEQ ID NO:4); and TaR1 (SEQ ID NO:3) polypeptides].

The polynucleotide sequence utilized by the method of the present invention can be ligated to appropriate regulatory elements to generate a nucleic acid construct. Preferably, the nucleic acid construct is an expression construct (i.e., an expression vector) which includes the polynucleotide sequence under the transcriptional regulation of a promoter functional in the host cell

Any suitable promoter sequence capable of directing transcriptional regulation of the exogenous polunucleotide in the host cell can be used by the nucleic acid construct of the present invention. Preferably, the promoter is selected from the group consisting of the tryptophan (trp) promoter, the lactose (lac) promoter, the T7 promoter, the lambda.-derived P_{L} promoter, or any of the promoters described in U.S. Pat. No. 6,410,301.

The nucleic acid construct of the present invention can further include an enhancer, which can be adjacent or distant to the promoter sequence and can function in up regulating the transcription therefrom.

In addition, the nucleic acid construct of the present invention may also include a nucleotide sequence encoding a signal for secretion of one or more polypeptides encoded by the exogenous polynucleotide to the outside of the host cell. Secretion signals generally contain a short sequence (7-20 residues) of hydrophobic amino acids. Secretion signals suitable for use in this invention are widely available and are well known in the art, see, for example by von Heijne [J. Mol. Biol. 184:99-105 (1985)] and by Lej et al., [J. Bacteriol. 169: 4379 (1987)].

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells. The construct according to the present invention can be, for example, a plasmid, a bacmid, a phagemid, a cosmid, a phage, a virus or an artificial chromosome.

The nucleic acid construct of the present invention can be utilized to stably or transiently transform host cells. In stable transformation, the nucleic acid construct of the present invention is integrated into the host cell genome and as such it represents a stable and inherited trait. In transient transformation, the nucleic acid construct is expressed by the cell transformed but it is not integrated into the genome and as such it represents a transient trait.

It should be appreciated that the host cell of the present invention can be transformed with one or more nucleic acid constructs, wherein each nucleic acid construct includes a polynucleotide sequence encoding one or more polypeptides participating in the synthesis of antibiotic TA.

Suitable methods of introducing nucleic acid constructs into prokaryotic and eukaryotic cells are well known in the art including, but not limited to, electroporation, protoplast transformation, calcium phosphate precipitation, calcium chloride treatment, microinjection, transfection by contact with a recombined virus, liposome-mediated transfection, DEAE-dextran transfection, transduction, conjugation, or microprojectile bombardment. Suitable transformation methods are described, for example, in Sambrook, J. and D. W. Russel “Molecular Cloning: A Laboratory Manual” 3^{rd} Edition, Cold Spring Harbor, 2001; and Glover, D. and B. D Hames “DNA Cloning: Core Techniques, Oxford University Press, 2002.

It will be appreciated that a host cell selected for transformation may be capable of expressing one or more endogenous polypeptides which are homologous to the polypeptides set forth in SEQ ID NOs: 1 and 3-19. In such a case, the expression construct of the present invention preferably does not include polynucleotide sequences encoding such polypeptides. However, in cases where the endogenous polypeptide homologues are incapable of effectively supporting the synthesis of antibiotic TA (e.g., due to low catabolic activity or wrong temporal expression), their expression or activity in the transformed cells is preferably downregulated.

Downregulating expression of a specific endogenous polypeptide in the host cell may be effected by administering the host cell to a small interfering RNA (siRNA) molecule. RNA interference is a two step process. the first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tusch1 ChemBiochem. 2:239-245 (2001); Cullen Nat. Immunol. 3:597-599 (2002); and Brant1 Biochem. Biophys. Act. 1575:15-25 (2002).

Synthesis of RNAi molecules suitable for use with the present invention can be effected as follows. First, the polypeptide mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Alternatively, downregulating expression of a specific endogenous polypeptide in the host cell can be effected by administering the host cell a DNAzyme molecule capable of specifically cleaving an mRNA transcript or DNA sequence of the polypeptide. DNAzymes are single-stranded polynucleotides which are capable of cleaving both single and double stranded target sequences (Breaker, R. R. and Joyce, G. Chemistry and Biology 1995;2:655; Santoro, S. W. & Joyce, G. F. Proc. Natl, Acad. Sci. USA 1997;943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

Alternatively, downregulation expression of a specific endogenous polypeptide in the host cell can be effected by using an antisense polynucleotide capable of specifically hybridizing with an mRNA transcript encoding the endogenous polypeptide.

Design of antisense molecules which can be used to efficiently downregulate of endogenous polypeptide must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

Alternatively, downregulating expression of a specific endogenous polypeptide in the host cell can be effected by administering the host cell a ribozyme molecule capable of specifically cleaving an mRNA transcript encoding the polypeptide. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

The transformed cells of the present invention are cultured under conditions suitable for synthesis of antibiotic TA. Preferably, the transformed cells are cultured in conventional fermentation bioreactor using methods well known in the art such as described, for example, in U.S. Pat. Nos. 6,214,221, 6,100,061, 5,998,184 and 5,571,720.

Alternatively, antibiotic TA antibiotic can be synthesized in a cell-free system. A suitable cell-free system includes the polypeptide or polypeptides of the present invention (obtained via secretion from the host cells or from lysing the host cells), appropriate buffer and substrates required for the synthesis of antibiotic TA. Methods of enzymatically synthesizing polyketides in cell-free systems are well known in the art and described, for example, in U.S. Pat. No. 6,531,229; Pieper et al., Nature 378: 263-266, 1996; Dimroth et al., Eur. J. Biochem. 13:98, 1970; Beck et al. Eur. J. Biochem. 192:487, 1990; Spencer et al. Biochem. J. 288:839, 1992; Lanz, et al. J. Biol. Chem. 266:9971, 1991; and Shen et al., Science 262:1535, 1993.

The antibiotic TA product can be isolated from the fermentation medium, or from the buffers utilized in cell-free systems, using a variety of standard protein recovering and purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. Suitable protein purification techniques are described, for example, in Rajni Hatti-Kaul and Bo Mattiasson (Eds) “Isolation and Purification of Proteins”, Biotechnology and Bioprocessing, Marcel Dekker (2003); and Rocky S. Tuan (Ed.) “Recombinant Protein Protocols: Detection and Isolation”, Methods in Molecular Biology, Vol 63, Humana Press (1997).

The present invention can also be utilized for producing a modified antibiotic TA. Thus, according to another aspect of the present invention, there is provided a method of producing a modified antibiotic TA which includes the steps of (i) mutating a polynucleotide sequence encoding at least one polypeptide selected from the group consisting of SEQ ID NOs: 1 and 3-19; (ii) expressing the mutated polynucleotide sequence in a host cell; and (iii) culturing the host cell under conditions suitable for synthesis of antibiotic TA, thereby producing the modified antibiotic TA.

Methods of producing novel polyketides, via mutation of polypeptides involved in polyketides biosynthesis, are described, for example, in U.S. Pat. Nos. 6,710,189 and by Kao et al. (J. Am. Chem. Soc. (1995) 117:9105-9106, 1996), Kao et al. (J. Am. Chem. Soc. 118:9184-9185, 1996), Donadio et al. (Science 252:675-679, 1991), Donadio et al. (Proc. Natl. Acad Sci. USA 90:7119-7123, 1993), Bedford et al. (Chem. Biol. 3:827-831, 1996), Oliynyk et al. (Chem. Biol. 3:833-839, 1996) and Kuhstoss et al. (Gene 183:231-236, 1996)

Accordingly, the polynucleotide sequence of the present invention, described hereinabove, can be mutated to encode altered polypeptide or polypeptides to thereby synthesize a modified antibiotic TA in the host cell. Mutation can be effected by using standard molecular biology techniques well known in the art such as described, for example, in Sambrook, J. and D. W. Russel (Eds.) “Molecular Cloning: A Laboratory Manual” 3^{rd} Edition, Cold Spring Harbor (2001). Preferably the mutation is effected by an insertion of one or more nucleotides, by deletion of one or more nucleotides, or by a substitution of one or more nucleotides using methods such as described, for example, in U.S. Pat. Nos. 6,642,027 and 6,461,839.

Expressing the mutated polynucleotide sequence in host cells, culturing the host cells and isolating the modfied antibiotic TA product can be effected using the methods described hereinabove for producing antibiotic TA.

The isolated modified TA product of the present invention can be tested for various clinically-related biological activities and such as, but not limited to, antimicrobial activities, anti-cancer activities, or immuno-suppression activities, using methods well known in the art (see, for example, Rosenberg et al., Agents Chemother. 4: 507-513, 1973). In addition, native or modified antibiotic TA generated using the methodology described herein can be tested for its efficacy in clinical applications such as gingivitis treatment (see, for example, Manor et al., J. Clin. Periodontol 16:621-624, 1989) and the like, in order to determine its commercial applicability.

Hence, the present invention provides novel methods of producing antibiotic TA, or derivatives thereof, using host cell expression of one or more polypeptides involved in TA synthesis.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### EXAMPLE 1

### Genes Encoding Polypeptides Involved in the Biosynthesis of Antibiotic TA

Materials and Methods:

Bacterial strains and plasmids: Myxococcus xanthus strains used were the wild-type strain ER-15 and transposition mutant strains ER-2514, ER-1037, ER-1030, ER-1311, ER-7513, ER-3708, ER-4639 and ER-6199, which are blocked in TA production (Varon et al., 1992). Escherichia coli strains TG1 (Bethesda Research Laboratories) and XL-1 Blue MR (Stratagene) were used for cloning and manipulating DNA. A conjugative tagged Tn1000 transposition system was used for sequencing using the procedure described in Sedgwick and Morgan (Methods in Mol. Genet. 3: 131-140). The vectors MH1578 and MH1599, pUC18, pUC19 (Norrander et al., Gene 26: 101-106, 1983) and SUPERCOS-1 (Stratagene) were used for both cloning and sequencing.

Media and growth conditions: E. coli was cultured at 37° C. in Luria broth (LB), or on LB agar, with the appropriate antibiotics (Sambrook et al., 1989). M. xanthus was cultured at 32° C. in 0±5 CTS, 1 CT or CTK medium, as required, or on media solidified with 1±5% Bacto agar (Difco) as described by Tolchinsky et al. (Antimicrob. Agents Chemother 36: 2322-2327, 1992; and Varon et al., 1992).

DNA sequencing and analysis: Automated DNA sequencing was performed on double-stranded DNA templates by the dideoxynucleotide chain-termination method (Sanger et al., Natl. Acad. Sci. USA 74: 5463-5467, 1977) using an Applied Biosystems model 373A sequencer. Sequence analysis for ORFs was carried out using the MacVector® 3.5 (International Biotechnologies) software.

Results:

Analysis of the antibiotic TA gene cluster: Chromosomal DNA was extracted from TA mutants ER-2514, ER-1037, ER-1030, ER-1311, ER-7513, ER-3708, ER-4639 and ER-6199 (Varon et al., 1992), then digested with restriction enzymes (which cut within the transposon) and analyzed by Southern hybridization with six different probes originating from TnV and Tn5lac and designed to hybridize either to the entire transposon, or to its 5′ or 3′ ends. A chromosomal restriction map of the entire TA gene cluster which was constructed on the basis of these results is illustrated in FIG. 1.

Preparation of antibiotic TA-specific probes: DNA from the TnV mutants ER-4639, ER1311 and ER-6199 was digested with KpnI (does not restrict TnV), self-ligated and transformed into E. coli XL1-Blue MR using the transposon-derived kanamycin resistance for selection. Tranformant clones pPYT4639, pPYT1311/p5 and pPYT6199 carried a 1.5 kb, 2.3 kb and a 11.2 kb fragment, respectively (see FIG. 1).

Cloning and characterizing DNA regions encoding polypeptides involved in antibiotic TA biosynthesis: A library of M xanthus ER-15 was constructed in the cosmid vector SUPERCOS-1 and screened using specific TA probes obtained from transposition mutants ER-4639, ER-1311 and ER-6199. Seventy four recombinant cosmids carrying genes required for antibiotic TA synthesis were identified through colony hybridization. The cosmids pPYCC64 and pPYCC44, which hybridized to these probes, were further characterized through restriction analysis (see FIG. 1), and subcloned for sequencing. The sequences of cloned inserts of these cosmids (designated regions 1 and 2, respectively) were determined as set forth in SEQ ID NOs: 2 and 20 (for regions 1 and 2, respectively). Computer analysis identified one ORF (ta1) transcribed by SEQ ID NO:2 and seventeen ORFs (taA, taB, aC, taD, taE, taF, taG, taH, taI, taJ, taK, taL, taM, taN, taR3, taR2 and taR1) transcribed by SEQ ID NO:20 (see Table 1 below). The deduced amino acid sequences and functions of the encoded polypeptides are presented in Table 2 below.

TABLE 1
DNA sequences isolated from the TA gene cluster
of Myxococcus xanthus

| SEQ ID NO. | Size (bp) | ORFs |
|---|---|---|
| 2 | 7,178 | ta1 |
| 20 | 19,053 | taA, taB, taC, taD, taE, taF, |
| | | taG, taH, taI, taJ, taK, taL, |
| | | taM, taN, taR3, taR2 and taR1 |

ORF—open reading frame

TABLE 2
Polypeptides encoded by the TA gene cluster of Myxococcus xanthus

| SEQ ID | |
|---|---|
| NO. | Function |
| 1 | Ta1 - synthetase unit and a PKS module |
| 3 | TaR1 - starvation response activator |
| 4 | TaR2 - σ⁵⁴ dependent Enhancer Binding Protein |
| 5 | TaR3 ammonium regulator/effector protein |
| 6 | TaA - NUS-G like transcription antiterminator |
| 7 | TaB - an ACP |
| 8 | TaC - beta-ketoacyl (ACP) synthase III (KAS III FabH) |
| 9 | TaD - membrane associated protein |
| 10 | TaE - an ACP |
| 11 | TaF - beta-ketoacyl (ACP) synthase III (KAS III FabH) |
| 12 | TaG - signal peptidase II (LSPA) |
| 13 | TaH - cytochrome P450 hydroxylase (cP450) |
| 14 | TaI - malonyl CoA (ACPP transacylase (MCT, FabD) |
| 15 | TaJ - malonyl CoA (ACPP transacylase (MCT, FabD) |
| 16 | TaK - 3-oxoacyl (ACP) synthase (KAS I, FabB) |
| 17 | TaL - enoyl CoA hydratase |
| 18 | TaM - enoyl CoA hydratase |
| 19 | TaN - O-methyltransferase (fragment) |

PKS—polyketide synthase
ACP—acyl carrier protein

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

### References

### (Additional References are Cited Hereinabove)

1. Rosenberg, E., Vaks, B. and Zuckerberg. A. Bactericidal action of an antibiotic produced by Myxococcus xanthus. Antimicrob. Agents. Chemother. 4:507-513 (1973).

2. Rosenberg, E., Porter, J. M., Nathan, P. N., Manor, A. and Varon, M. Antibiotic TA: an adherent antibiotic. Bio/Technology. 2:796-799 (1984).

3. Varon et al., Mutation and mapping of genes involved in production of the antibiotic TA in micrococcus xanthus. Antimicrob. Agents Chemother. 36: 2316-2321 (1992).

4. Marshak et al, “Strategies for Protein Purification and Characterization. A laboratory course manual.” CSHL Press, 1996.

5. Testoni et al, 1996, Blood 87:3822.

6. PCR Protocols: A Guide To Methods And Applications, Academic Press, San Diego, Calif. (1990).

7. Sambrook et al., Molecular Cloning: A Laboratory Manual, Cold Springs Harbor Laboratory, New York (1989, 1992).

8. Ausubel et al., Current Protocols in Molecular Biology, John Wiley and Sons, Baltimore, Md. (1989).

What is claimed is:

1. A method of producing an antibiotic TA comprising:
(a) expressing in a host cell an exogenous polynucleotide sequence encoding at least one polypeptide selected from the group consisting of SEQ ID NOs: 1 and 3-19; and
(b) culturing said host cell under conditions suitable for synthesis of the antibiotic TA, thereby producing the antibiotic TA.

2. The method of claim 1, wherein said expressing is effected by transforming said host cell with a nucleic acid construct including said exogenous polynucleotide under the transcriptional control of a promoter functional in said host cell.

3. The method of claim 2, wherein said nucleic acid construct further includes a nucleotide sequence encoding a signal for secretion of said at least one polypeptide to the outside of said host cell.

4. The method of claim 1, further comprising regulating an expression or activity of at least one endogenous polypeptide capable of modulating said synthesis of said antibiotic TA.

5. The method of claim 1, further comprising:
(c) isolating said antibiotic TA produced in said host cell.

6. The method of claim 1, wherein said host cell is a eukaryotic or a prokaryotic host cell.

7. The method of claim 6, wherein said prokaryotic host cell is E. coli.

8. The method of claim 6, wherein said prokaryotic host cell is a Myxococcus species.

9. The method of claim 8, wherein said wherein said Myxococcus species is Myxococcus xanthus.

10. A method of producing a modified antibiotic TA, comprising:
(a) mutating a polynucleotide sequence encoding at least one polypeptide selected from the group consisting of SEQ ID NOs: 1 and 3-19;
(b) expressing a mutated polynucleotide sequence resulting from step (a) in a host cell; and
(c) culturing said host cell under conditions suitable for synthesis: of antibiotic TA, thereby producing the modified antibiotic TA.

11. The method of claim 10, wherein said mutation is effected by a deletion of one or more nucleotides.

12. The method of claim 10, wherein said mutation is effected by an insertion of one or more nucleotides.

13. The method of claim 10, wherein mutation is effected by a substitution of one or more nucleotides.

14. The method of claim 10, wherein said expressing is effected by transforming said host cell with an expression vector including said mutated polynucleotide under the transcriptional regulation of a promoter functional in said host cell.

15. The method of claim 10, wherein said expressing is effected by transforming said host cell with a nucleic acid construct including said mutated exogenous polynucleotide under the transcriptional control of a promoter functional in said host cell.

16. The method of claim 13, wherein said nucleic acid construct further includes a nucleotide sequence encoding a signal for secretion of said at least one polypeptide to the outside of said host cell.

17. The method of claim 9, further comprising regulating an expression or activity of at least one endogenous polypeptide capable of modulating said synthesis of said modified antibiotic TA in said host cell.

18. The method of claim 9 further comprising isolating said modified antibiotic TA.

19. The method of claim 9, wherein said host cell is a eukaryotic or a prokaryotic host cell.

20. The method of claim 19, wherein said prokaryotic host cell is E. coli.

21. The method of claim 19, wherein said prokaryotic host cell is a Myxococcus species.

22. The method of claim 21, wherein said Myxococcus species is Myxococcus xanthus.
